(19) United States
(12) Patent Application Publication
(10) Pub. No.: US 2023/0181249 A1
(43) Pub. Date: Jun. 15, 2023

(54) CONNECTING DEVICE FOR SURGICAL INSTRUMENTS AND SURGICAL INSTRUMENT AND METHOD

(71) Applicant: KARL STORZ SE & Co. KG, Tuttlingen (DE)

(72) Inventor: Fridolin PREIS, Tuttlingen (DE)

(21) Appl. No.: 18/071,725

(22) Filed: Nov. 30, 2022

(30) Foreign Application Priority Data
Dec. 9, 2021 (DE) 10 2021 132 426.2

(51) Int. Cl. A61B 18/14

(52) U.S. Cl. CPC A61B 18/1492; A61B 2018/00172

(57) ABSTRACT

A connection device for surgical instruments includes a housing, a head part coupling which is arranged at the housing and is configured for a mechanical coupling of a head part. The head part coupling includes a pin element, which is configured such that the head part can be fixed to the housing in a non-displaceable manner. The pin element is fixed non-detachably to the housing via a weld seam. A surgical instrument includes the connection device and an accessory, which is guided through the head part. A process fixes the connection device with the pin element non-detachably fixed to the housing by a laser welding process.

### CROSS REFERENCE TO RELATED APPLICATIONS

This application claims the benefit of priority under 35 U.S.C. § 119 of German Application 10 2021 132 426.2, filed Dec. 9, 2021, the entire contents of which are incorporated herein by reference.

### FIELD OF THE INVENTION

The present invention pertains to a connection device for surgical instruments, as well as a surgical instrument and a process.

### TECHNICAL BACKGROUND

Surgical instruments often have a connection device and a guide element for an accessory, which guide element can be attached to the connection device by means of a head part coupling. The guide element forms a head part, which is arranged at the distal end of the surgical instrument.

In high-frequency surgery, the thermal effect of high-frequency alternating current on tissue is used in so-called electrotomy (also diathermy or electrocautery and in coagulation to cut target tissue and possibly to stop bleeding at the same time or instead of this. The surgical instruments have for this purpose one or more (active) electrodes at the surgical instrument accessories, via which electrodes the high-frequency alternating current is introduced into the target tissue of a patient. A high current density is introduced at the target tissue so that the desired effect of electrotomy and/or coagulation occurs at the target tissue. Therefore, (active) electrodes usually have a small surface and have, for this purpose, for example, a needle-shaped or blade-shaped configuration in order to introduce the applied high-frequency alternating current into the target tissue with the highest possible current density. Either monopolarly or bipolarly operated surgical instruments may be used in this case.

A single active electrode, via which the high-frequency alternating current is introduced into the tissue of a patient, is arranged at the accessory of the surgical instrument in case of monopolarly operated surgical instruments. A large-surface counterelectrode (neutral electrode) must be attached to the body of the patient as an opposite pole so that the current is introduced from the single active electrode into the tissue.

Two electrodes (active electrode and neutral electrode) are arranged at the accessory of the surgical instrument in case of bipolarly operated surgical instruments. In this connection, the high-frequency alternating current is introduced into the target tissue from a first electrode (active electrode) directly opposite the second electrode (neutral electrode).

A monopolar or bipolar surgical instrument may be configured as a resectoscope, especially for transurethral resection. An optimized incision characteristic and a precise cutting or coagulation quality must be ensured during the entire procedure in case of a bipolar resectoscope.

The head part, especially in a resectoscope, is arranged at a shaft of the surgical instrument and forms the frontal or distal element, which is used for guiding the surgical instrument through a section of the body. For this purpose, the head part must be configured as sufficiently stable and durable. A variety of accessories, for example, a cold snare, a diagnostic hysteroscope or high-frequency electrodes can be held by the head part for different indications. Since the head part is arranged directly at the point stressed by a force, this head part must be fastened to the shaft in a stable manner. A fixing of the head part at the shaft may be provided, for example, by a bonded connection.

### SUMMARY OF THE INVENTION

Against this background, a basic object of the present invention is to provide an improved connection device for surgical instruments, especially bipolarly operable surgical instruments.

This object is accomplished by a connection device having features according to the present invention as well as by a surgical instrument having features according to the present invention and a process having features according to the present invention.

Accordingly, provisions are made for:
A connection device for surgical instruments, with a housing, and with a head part coupling, which is arranged at the housing and is configured for the mechanical coupling of a head part, wherein the head part coupling has a pin element, which is configured such that the head part can be fixed to the housing in a non-displaceable manner, wherein the pin element is fixed non-detachably to the housing via a weld seam.
A surgical instrument, especially for high-frequency surgery, with a connection device according to the present invention.
A process for fixing a connection device according to the present invention, wherein the pin element is non-detachably fixed to the housing by means of a laser welding process.

The underlying principle of the present invention is to fix the head part non-detachably and securely to the housing, especially to a shaft of the instrument, by means of a positive-locking connection in combination with a weld seam.

The surgical instrument may be configured especially as a monopolarly and/or bipolarly operable surgical instrument. Especially in high-frequency surgery, tissue can be cut (electrotomy) and, in addition or as an alternative, bleeding can be stopped (coagulation) using the surgical instrument, which can be operated in a monopolar or bipolar manner, as desired. The monopolarly and bipolarly operable surgical instrument may be, for example, an instrument guided manually by a user or an operator (e.g., surgeon) or an instrument guided by a robot (arm).

The housing may have an integral or one-piece configuration or may be formed from a plurality of components (e.g., shell elements), which are mechanically connected to one another detachably or permanently. The housing may be at least partially manufactured from a plastic, from a metal or from an alloy. The housing may preferably be at least partially manufactured from a biocompatible material.

The housing may comprise a shaft, which is non-detachably and securely fixed to the head part via the head part coupling.

The housing has a tubular configuration especially at least in an area, in which the housing is in connection with the head part, and may especially have a diameter in the range of 4 mm to 10 mm, for example, 5 mm.

The shaft is preferably configured for the at least partial mounting of the head part. For example, the shaft may form a kind of outer tube for the head part. The head part may especially be inserted with a partial section into the shaft to orient the head part and to bring the head part to the geometrically desired position. Therefore, especially the non-detachable fixing of the head part to the shaft takes place via the connection device. The connection device is configured such that a head part mechanically connected thereto can be held securely.

The head part coupling has a pin element, which forms a positive-locking connection between the housing, especially the shaft, and the head part. In particular, the pin element has a small configuration in relation to a diameter of the head part or of the housing. The head part may be configured for guiding an accessory.

The head part is especially a ceramic insert, which is inserted at a distal end of the shaft and is fixed in a positive-locking manner via the pin element.

The weld seam is preferably produced by a laser welding process, so that a weld seam that is precise and necessary for a desired tolerance range can be formed.

The head part can be non-detachably fixed to the housing in a rapid manner and at a low cost using the connection device according to the present invention, so that a falling out of the head part during the surgery and a remaining of the head part in the patient can be reliably avoided.

Advantageous embodiments and variants appear from this disclosure including from the description with reference to the figures of the drawings.

According to an advantageous embodiment the pin element can extend through the housing through a hole and mesh with a formation (recess/bulge) in the head part. Advantageously, a diameter of the hole is adapted to a diameter of the pin element, so that the pin element can be securely welded to the housing. In particular, the pin element closes flush with an outer surface of the housing, so that the surgical instrument is not affected by the connection device. The formation in the head part may be configured as a depression or as a pot shape mount. Hence, the pin element is preferably held within the formation and cannot pierce the head part.

According to one variant, the pin element may connect the head part to the housing in a positive-locking manner. In this way, a displacement of the head part relative to the housing is prevented, as a result of which the head part can be held in a position-fixed manner.

According to one embodiment, the housing may have a tubular configuration. Advantageously, an at least partial insertion of the head part into the housing is possible in this manner. The housing preferably forms a shaft, which may have a diameter of, for example, 5 mm to 10 mm. Diameters that are less than 5 mm are, furthermore, conceivable. The available wall thickness of the tubular housing is therefore very small, so that a welding together of the pin element with the housing preferably takes place by means of laser welding because of the low tolerance threshold.

According to an advantageous embodiment the head part may have a tubular configuration. In such an embodiment, the housing advantageously likewise has a tubular configuration. In this case, the housing may form a kind of outer tube, into which a partial area of the head part is inserted. The diameters of the head part and of the outer tube are preferably adapted to one another, so that only a small gap forms.

According to an advantageous embodiment, an accessory may be able to be guided through the head part. In particular, a monopolarly and/or bipolarly operable accessory may be able to be guided through the head part. This is especially made possible by the head part forming a hollow cross section. The head part coupling, i.e., especially the pin element, does not have an effect on the area that is used for guiding through the accessory (does not block or otherwise interfere with the accessory being guided through the head part.

According to a preferred embodiment the pin element may be oriented transversely to a longitudinal axis of the housing. A positive-locking connection is advantageously possible in this manner, since the pin element is oriented transversely to a loading direction during an insertion or a removal of the surgical instrument into or out of the body region.

According to a preferred embodiment, the head part may contain ceramic or be made of ceramic. In this way, the embodiment of an especially stable as well as durable head part is possible. Thermal damage to the head part, due to the inclusion of a ceramic in the head part, can especially be prevented.

According to an especially preferred embodiment, the connection device may be configured for a resectoscope. In particular, it may be a 5-mm resectoscope. Because of the resectoscope very small diameter, such a resectoscope makes it possible to treat outpatient infertility patients as well as patients with bleeding disorders without anesthesia. Hence, only a very small dilation or no dilation is necessary. In particular, a bipolar system can be provided without return of the current flow over the shaft, i.e., over the housing. The head part is preferably configured as a ceramic insert and can be used, for example, for cold snares, as a diagnostic hysteroscope as well as for high-frequency electrodes for different indications. An effective resection, coagulation or vaporization can be achieved thereby.

The same features and advantages, as described with regard to the connection device, apply to the surgical instrument according to the present invention as well as to the process for fixing the connection device, so that they will not be described in detail below.

The above embodiments and variants can, insofar as meaningful, be combined with one another as desired. Other possible embodiments, variants and implementations of the present invention also do not comprise explicitly mentioned combinations of features of the present invention, which features are described above or below with regard to the exemplary embodiments. In particular, the person skilled in the art will in this case also add individual aspects as improvements or additions to the respective basic form of the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

The present invention will be explained in more detail below based on the exemplary embodiments mentioned in the schematic figures of the drawings. In the drawings:

FIG. 1 is a side view and enlarged view showing an exemplary embodiment of a surgical instrument with a head part and with an accessory guided in the head part; and

FIG. 2 is a side view and enlarged view showing an exemplary embodiment of a surgical instrument during the welding of the head part coupling.

The attached figures of the drawings shall convey a further understanding of the embodiments of the present invention. They illustrate embodiments and are used in conjunction with the description for explaining the principles and concepts of the present invention. Other embodiments and many of the mentioned advantages appear in regard to the drawings. The elements of the drawings are not necessarily shown on a scale true to one another.

Identical, functionally identical and similarly acting elements, features and components are, unless stated otherwise, each provided with the same reference numbers in the figures of the drawings.

### DESCRIPTION OF EXEMPLARY EMBODIMENTS

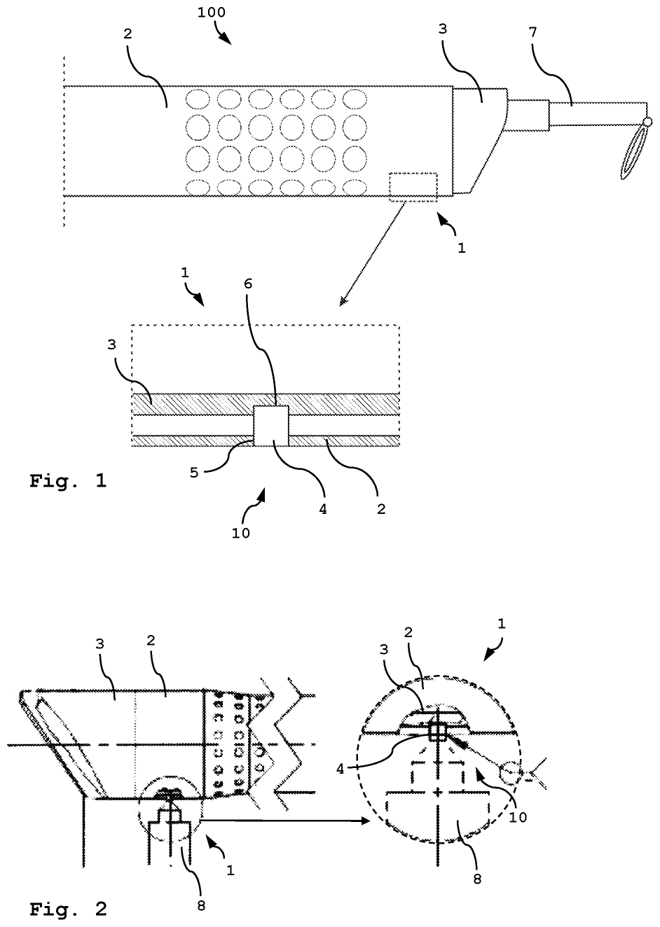

Referring to the drawings, FIG. 1 shows an exemplary embodiment of a surgical instrument 100 with a head part 3 and with an accessory 7 guided in the head part 3. Only a front area of the surgical instrument 100, in which front area a partial area of the housing 2 can be seen, is shown. The head part coupling 10 is arranged at a distal end of the surgical instrument 100 or of the housing 2. The head part coupling 10 is configured for the non-detachable mechanical connection of the head part 3 to the housing 2.

The housing 2 forms a shaft, which is configured for the mounting of a head part 3. An accessory 7 that projects over the head part 3 at the distal end is guided through the housing 2 as well as through the head part 3. In the drawing of FIG. 1, the accessory 7 is configured as a cold snare. It is also conceivable to arrange as an accessory 7 a diagnostic hysteroscope, optionally with 5 Fr. instruments, or a high-frequency electrode for different indications, as well as other instruments.

The accessory is a bipolarly operable accessory here. Of course, a monopolarly or a monopolarly and bipolarly operable accessory may also be connected to the surgical instrument 100.

The head part 3 is especially configured as a ceramic insert and is dimensionally stable and durable as a result.

The connection device 1 is shown in detail as an enlarged detail. The enlarged detail shows a sectional view, so that the meshing of the head part 3 with the housing 2 can be seen. The head part coupling 10 has a pin element 4, which forms a positive-locking connection between the housing 2 and the head part 3. The pin element 4 is guided through a hole 5 in the housing 2 and is held in a formation (recess) 6 in the head part 3. As a result, the pin element 4 cannot pierce the head part 3. Therefore, the pin element 4 is held non-displaceably in a radial direction.

The pin element 4 forms especially a local, i.e., punctiform, fixation and as a result needs little space for installation. The pin element 4 is oriented transversely to a longitudinal axis of the housing 2. In particular, the area without the head part, which is configured as a hollow cross section, is not affected by the pin element 4. Furthermore, the pin element 4 closes flush with an outer surface of the housing 2.

The housing 2 has a tubular configuration in a front area of the surgical instrument 100. The head part 3 likewise has a tubular configuration, wherein a diameter of the head part 3 or of the housing 2 may be in a range of 5 mm. Therefore, a very precise welding process is necessary for preparing a weld seam, which fixes the pin element 4 to the housing 2.

FIG. 2 shows a welding tool 8 for preparing such a weld seam. The weld seam is preferably prepared by means of a laser welding process, so that very low tolerances can be implemented. The weld seam may be configured as a continuous seam. In this case, for example, a V seam, an HV seam or a Y seam may be formed. Other weld seam shapes are likewise conceivable.

A detachment of the head part 3 and a remaining portion of the head part 3 in a patient can be effectively avoided due to the positive locking between the head part 3 and the housing 2 in conjunction with the weld seam, and in particular compared to a bonded connection from the state of the art. The connection device 1 can, furthermore, be embodied in a cost-effective and rapid manner, and thus likewise in a cost-effective manner compared to an embossing of both components.

Even though the present invention was described in full above on the basis of preferred exemplary embodiments, it is not limited thereto, but can be modified in a variety of ways.

For example, the head part 3 may also be guided outside of the housing 2. In such an embodiment, the hole 5 would be formed in the head part 3 and the formation 6 would be formed in the housing 2. In another embodiment, not shown, the connection device 1 may have two or more head part couplings 10, which are arranged along the circumference of the housing 2 or of the head part 3 in the front area of the surgical instrument 100. The stability of the connection can be increased further as a result.

While specific embodiments of the invention have been shown and described in detail to illustrate the application of the principles of the invention, it will be understood that the invention may be embodied otherwise without departing from such principles.

### LIST OF REFERENCE NUMBERS

1 Connection device
2 Housing
3 Head part
4 Pin element
5 Hole
6 Formation (Recess/Bulge)
7 Accessory
8 Welding tool
10 Head part coupling
100 Surgical instrument

1. A connection device for surgical instruments, comprising:
a housing;
a head part;
a head part coupling, which is arranged at the housing and is configured for a mechanical coupling of the head part, wherein the head part coupling comprises a pin element, which is configured to non-dispacelably fix the head part to the housing, and a weld seam, wherein the pin element is fixed non-detachably to the housing via the weld seam.

2. A connection device in accordance with claim 1, wherein the pin element extends through the housing through a hole and meshes with a formation in the head part.

3. A connection device in accordance with claim, wherein the pin element connects the head part to the housing with a positive-locking connection.

4. A connection device in accordance with claim 1, wherein the housing and/or the head part have a tubular configuration.

5. A connection device in accordance with claim 1, wherein the head part is configured such that an accessory can be guided through the head part.

6. A connection device in accordance with claim 1, wherein the pin element is oriented transversely to a longitudinal axis of the housing.

7. A connection device in accordance with claim 1, wherein the head part contains ceramic or is made of ceramic.

8. A connection device in accordance with claim 1, wherein the connection device is configured for a resectoscope.

9. A surgical instrument comprising:
a connection device comprising:
a housing;
a head part; and
a head part coupling, which is arranged at the housing and is configured for a mechanical coupling of the head part, wherein the head part coupling comprises a pin element, which is configured to non-dispacelably fix the head part to the housing, and a weld seam, wherein the pin element is fixed non-detachably to the housing via the weld seam; and
an accessory, which is guided through the head part.

10. Process for fixing a connection device, the process comprising the steps of:
providing the connection device, wherein the connection device comprises: a housing;
and a head part coupling, which is arranged at the housing and is configured for a mechanical coupling of a head part, wherein the head part coupling comprises a pin element, which is configured to non-dispaceably fix the housing to the head part, and a weld seam, wherein the pin element is fixed non-detachably to the housing via the weld seam;
fixing the pin element non-detachably to the housing by means of a laser welding.

11. A process in accordance with claim 10, further comprising configuring the pin and the head part such that the non-dispaceable fixation of the housing to the head part is comprised by a positive-locking connection of the head part to the housing.

12. A process in accordance with claim 10, wherein the housing and the head part have a tubular configuration.

13. A process in accordance with claim 12, wherein the head part is configured to guide an accessory through the head part.

14. A surgical instrument in accordance with claim 9, wherein the pin element extends through the housing through a hole and meshes with a formation of the head part.

15. A surgical instrument in accordance with claim 9, wherein the pin element connects the head part to the housing with a positive-locking connection.

16. A surgical instrument in accordance with claim 9, wherein the housing and/or the head part have a tubular configuration.

17. A surgical instrument in accordance with claim 9, wherein the accessory comprises a monopolarly and/or bipolarly operable accessory.

18. A surgical instrument in accordance with claim 9, wherein the pin element is oriented transversely to a longitudinal axis of the housing.

19. A surgical instrument in accordance with claim 9, wherein the head part comprises a ceramic.

20. A surgical instrument in accordance with claim 9, wherein the connection device is configured for a resectoscope.
